(19) United States
(12) Patent Application Publication
(10) Pub. No.: US 2021/0163813 A1
(43) Pub. Date: Jun. 3, 2021

(54) ETHYLENEAMINE SULFONATE-BASED SURFACTANT FOR HIGH TEMPERATURE FOAMING

(71) Applicant: Dow Global Technologies LLC, Midland, MI (US)

(72) Inventors: Wenke Miao, Shanghai (CN); Peng Gao, Shanghai (CN); Lixin You, Shanghai (CN); Cheng Shen, Shanghai (CN); Pramod D. Patil, Sugar Land, TX (US); Biplab Mukherjee, Pearland, TX (US); Troy E. Knight, Missouri City, TX (US)

(21) Appl. No.: 16/954,066

(22) PCT Filed: Dec. 18, 2017
(86) PCT No.: PCT/CN2017/116874

§ 371 Date: Jun. 18, 2020

(51) Int. Cl. C09K 8/584; C07C 309/69; C07C 233/38; C07C 233/36; C07C 231/14; C07C 303/28; E21B 43/16

(52) U.S. Cl. CPC C09K 8/584; C07C 309/69; C07C 233/38; E21B 43/16; C07C 231/14; C07C 303/28; C07C 233/36

(57) ABSTRACT

Surfactants constructed from three synthetic building blocks that contain multiple hydrocarbon chains, ethyleneamine, and alkyl sulfonate salt groups, were shown to possess good thermal stability, and foamability, and high foam profiles. The materials are targeted for high temperature foaming applications, such as foam flooding enhanced oil recovery to improve conformance control and other oil and gas downhole foaming applications.

### FIELD OF THE INVENTION

This invention relates to surfactants and methods of synthesizing surfactants. More particularly, it relates to surfactants, methods of synthesizing surfactants, and treating crude oil with surfactants in enhanced oil recovery applications.

### BACKGROUND OF THE INVENTION

Surfactants are compounds that lower the surface tension between two liquids or between a liquid and a solid, and may act as detergents, wetting agents, emulsifiers, foaming agents, and dispersants. Surfactants are heavily utilized in oil and gas applications such as enhanced oil recovery, as well as home and personal care, industrial cleaning, and other industry applications. In chemical injection enhanced oil recovery applications, dilute solutions of surfactants such as petroleum sulfonates or biosurfactants such as rhamnolipids may be injected to lower the interfacial tension or capillary pressure that impedes oil droplets from moving through a reservoir. Application of these methods is usually limited by the cost of the chemicals and their adsorption and loss onto the rock of the oil containing formation.

In particular, high stable foaming surfactants have been shown to be a very effective technology for improving gas conformance in the reservoir. The general definition of a foam is a gas (e.g. air, CO₂, N₂, hydrocarbon, steam) dispersed in a liquid. Foams destabilize due to lamella drainage, capillary pressure effects, and weakness of foaming agent properties, such as surface elasticity, surface rheology, and interaction with liquid.

High stable foaming surfactants are known in the art. U.S. Pat. No. 5,914,310 and EP0697245 disclose an amphoteric surfactant containing at least two hydrophobic chains and at least two hydrophilic chains per molecule. Similarly, WO/1998/15345 discloses aqueous surfactant compositions comprising a surfactant mixture with one or more gemini surfactants that further comprises at least two hydrophobic chains, and at least two hydrophilic chains.

Separately, U.S. Pat. No. 3,703,535 discloses an amphoteric surface active agent with hydrophilic hydroxyl groups, while U.S. Published Patent Application No. 2006/0247324 discloses amphoteric surfactants derived from ethyleneamines with COOH or SO₃H hydrophilic groups for use in treating paper, fibers, textiles, hair, and human skin. Finally, JP10204475 discloses an anionic surfactant having a specific two-chain, monopolar group, for use in hair conditioning.

In addition to these references, it has been shown in the art that improvement in foam stability can be achieved with a water soluble polymer to increase viscosity, or alkyl alcohol to strengthen the surfactant interaction with water, however these additives increase cost. What is needed is a low-cost means of improving foam stability that can be utilized in enhanced oil recovery applications.

### SUMMARY OF THE INVENTION

The invention is a surfactant for high temperature foaming that can be used in enhanced oil recovery applications. The structure of the surfactant comprises an ethyleneamine backbone with two or more linear hydrophobic tails as well as at least one sulfonate salt hydrophilic group. The surfactant can be used alone as the primary foamer or as a co-surfactant with other foaming surfactants such as anionic surfactants such as alpha olefin sulfonates (AOS) and internal olefin sulfonates (IOS), as well as nonionic alkoxylate surfactants, cationic surfactants, or blends of anionic and nonioinic foaming agents.

Because of the novel surfactant's strong intermolecular interaction, including multiple hydrogen bonds and/or multiple hydrophobic interactions, interactions between surfactants can be promoted, which enhances foam stability significantly. Moreover, because the components of the novel surfactant are widely available and require little effort to produce, the surfactant can be prepared at a low-cost.

### BRIEF DESCRIPTION OF THE FIGURES

FIG. 1 is an example equation for the synthesis of the surfactants DETA-C10-SS and TETA-C10-SS.

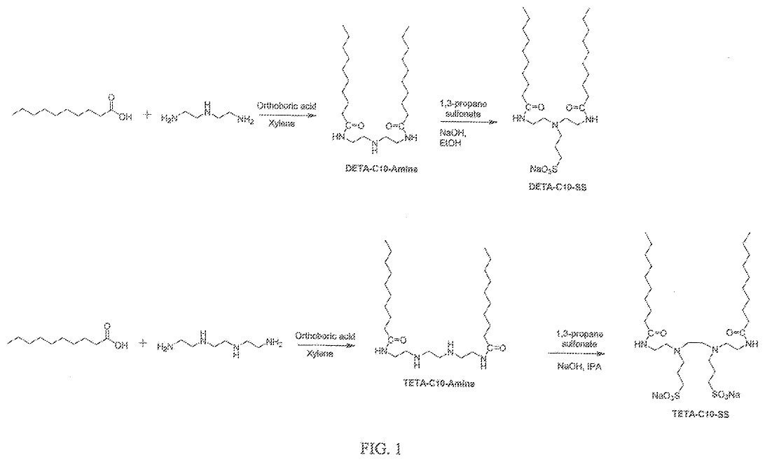

FIG. 2 is a chart depicting Thermogravimetric Analysis (“TGA”) data for novel DETA-C10-SS and TETA-C10-SS surfactants in both air and N₂ atmospheres.

FIG. 3 is a chart depicting data obtained from Ross-Miles testing of DETA-C10-SS and TETA-C10-SS at room temperature.

FIG. 4 is a chart depicting Foam Scan data for DETA-C10-SS and TETA-C10-SS at room temperature.

FIG. 5 is an image depicting foam morphology of DETA-C10-SS (A) and TETA-C10-SS (B) at room temperature.

FIG. 6 is a chart depicting foam scan data of DETA-C10-SS and TETA-C10-SS in 12.0% brine water at 80° C.

### DETAILED DESCRIPTION OF THE PREFERRED EMBODIMENTS

A representative synthesis reaction for the surfactant of the invention is as follows:

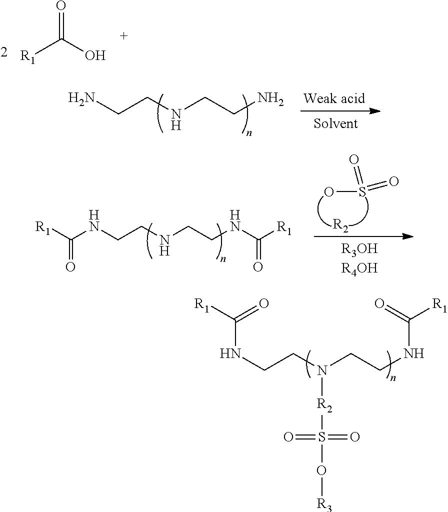

wherein R₁ is a C₅-C₂₁ linear or branched, saturated or unsaturated alkane; R₂ is a C₃-C₁₀ linear or branched, saturated or unsaturated alkane; R₃ is Na, K, Ca, or Mg; R₄ is a C₂-C₃ linear or branched, saturated or unsaturated alkane; and n ≥1. Preferred solvents include, but are not limited to xylene. As the above reaction shows, the novel surfactant is synthesized by reacting an ethylene amine with an acid in solvent to form a solution, and then further treating the solution with a weak acid to form an intermediate amide. The intermediate amide is then reacted with a sultone, an inorganic alkali, and an alcohol as solvent, resulting in the novel surfactant.

Alternate methods of synthesizing the novel surfactant via an intermediate amine are as follows:

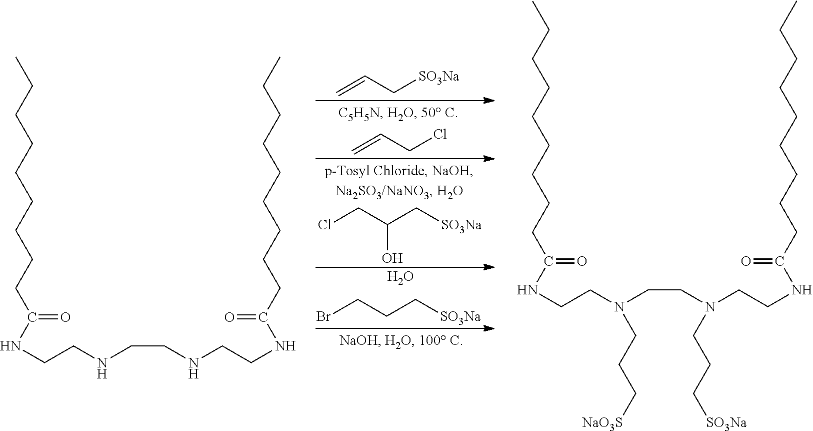

### Ethyleneamine

The surfactant of the invention utilizes as a precursor an ethyleneamine having the generic formula:

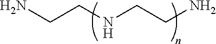

wherein n ≥1. Ethyleneamines are the preferred backbone for the surfactant of the invention as these molecules have more than two active sites (NH₂ and/or NH) that allow for amide functional groups in the system. The amide groups readily form intermolecular or intramolecular hydrogen bonds to stabilize surfactants along the foam bubble surface. Preferred ethyleneamines for the novel surfactant include Diethylenetriamine (DETA), Triethylene tetramine (TETA), Tetraethylene pentamine (TEPA), Pentaethylene hexamine (PEHA), Hexaethylene heptamine, (HEHA) and mixtures thereof. Preferably, ethyleneamines with a molecular weight between approximately 100 g/mol. and approximately 325 g/mol. are utilized in the surfactant synthesis reaction.

### Fatty Acid

In addition to ethyleneamines, the surfactant of the invention also utilizes as a precursor fatty acids having the following chemical formula:

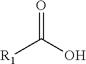

wherein R₁ is a C₅-C₂₁ linear or branched, saturated or unsaturated alkane. As the above formula shows, fatty acids are carboxylic acids with long unbranched aliphatic chains, which, when synthesized with ethyleneamines, result in hydrophobic tails for the surfactant of the invention. Preferred fatty acids for the synthesis of the surfactant of the invention include, but are not limited to, capric acid. Preferably, fatty acids with a molecular weight between approximately 100 g/mol. and approximately 300 g/mol. are utilized in the surfactant synthesis reaction.

### Weak Acid

A weak acid is utilized as a catalyst to facilitate amidation reaction of the fatty acid and ethyleneamine to form the surfactant precursor. Preferred weak acids include, but are not limited to, orthoboric acid. Preferably, approximately 0.01 wt. % to approximately 2.0 wt. % of weak acid is utilized in the surfactant synthesis reaction.

### Sulfonate Salt

The surfactant of the invention incorporates a sulfonate salt. The sulfonate salt is incorporated in the novel surfactant by reacting the intermediate amide with a sultone having a R₂ carbon chain, an inorganic compound having the formula R₃OH, and an alcohol having the formula R₄OH, wherein R₂ is a C₃-C₁₀ linear or branched, saturated or unsaturated alkane; R₃ is Na, Ca, or Mg; and R₄ is a C₂-C₃ linear or branched, saturated or unsaturated alkane. The alcohol may be a primary or secondary alcohol. Preferably, approximately 5.0 wt. % to approximately 35.0 wt. % of R₂-sultone; approximately 2.0 wt. % to approximately 12.0 wt. % of R₃OH, and approximately 30.0 wt. % to approximately 70.0 wt. % of R₄OH are utilized in the surfactant synthesis reaction.

### Alternate Methods

The surfactant of the invention may also be synthesized by reacting the intermediate amide with a compound having the chemical formula:

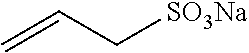

along with a C₅H₅N reagent, and utilizing H₂O as a solvent, at 50° C. Preferably, approximately 10.0 wt. % to approximately 70.0 wt. % of the compound; approximately 0.1 wt. % to approximately 10.0 wt. % of the C₅H₅N reagent and approximately 10.0 wt. % to approximately 90.0 wt. % of the H₂O solvent are utilized in this alternate synthesis reaction.

The surfactant of the invention may also be synthesized by reacting the intermediate amide with a compound having the chemical formula:

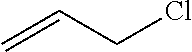

along with p-Tosyl Chloride, a reagent comprising a mixture of NaOH, Na₂SO₃, and NaNO₃, and utilizing H₂O as a solvent. Preferably, approximately 5.0 wt. % to approximately 50.0 wt. % of the compound; approximately 1.0 wt. % to approximately 30.0 wt. % of p-Tosyl Chloride, approximately 1.0 wt. % to approximately 20.0 wt. % of the NaOH reagent, approximately 0.1 wt. % to approximately 15.0 wt. % of the Na₂SO₃ reagent, approximately 0.1 wt. % to approximately 15.0 wt. % range of the NaNO₃, reagent, and approximately 10.0 wt. % to approximately 90.0 wt. % of the H₂O solvent are utilized in this alternate synthesis reaction.

The surfactant of the invention may also be synthesized by reacting the intermediate amide with a compound having the chemical formula:

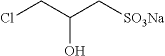

utilizing H₂O as a solvent. Preferably, approximately 15 wt. % to approximately 85 wt. % of the compound and approximately 10.0 wt. % to approximately 90.0 wt. % of the H₂O solvent are utilized in this alternate synthesis reaction.

The surfactant of the invention may also be synthesized by reacting the intermediate amide with a compound having the chemical formula:

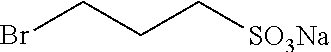

along with a NaOH reagent and utilizing H₂O as a solvent, at 100° C. Preferably, approximately 20.0 wt. % to approximately 85.0 wt. % range of the compound; approximately 5.0 wt. % to approximately 50.0 wt. % of the NaOH reagent and approximately 10.0 wt. % to approximately 90.0 wt. % of the H₂O solvent are utilized in this alternate synthesis reaction.

### Method of Use

Both the novel surfactant and intermediate amide may be utilized for oilfield applications, especially enhanced oil recovery processes such as foam flooding. A dilute solution of the novel surfactant, or a mixture of the novel surfactant and intermediate amide, is injected into a crude oil reservoir to lower the interfacial tension or capillary pressure that impedes the crude oil from moving through the reservoir. The novel surfactant can be utilized in concentrations of 5.0 ppm to 50,000 ppm, while the intermediate amide can be used in concentrations of 0.0 ppm to 50,000 ppm. The enhanced foaming properties of the novel surfactant and intermediate amide allow the substances to be used in environments where the temperature is up to 300° C. and the salinity is up to 20.0 wt. %. The novel surfactant and intermediate amide can be used alone as the primary foamer or as a co-surfactant with other foaming surfactants such as anionic surfactants—alpha olefin sulfonates (AOS) and internal olefin sulfonates (IOS), nonionic alkoxylate surfactants, cationic surfactants, or blends of anionic and nonioinic foaming agents.

### Working Examples

The following Examples illustrate various representative attributes of the invention but should in no way be construed as limiting.

Synthesis of DETA-C10-Amine Intermediate

DETA (1.0 eq) was dissolved in xylene, then capric acid (1.05 eq) (also dissolved in xylene) was added into the DETA-xylene solution drop by drop at a temperature of 60° C. When completed, orthoboric acid (0.005 eq) was added into the mixture and stirred at a temperature of 100° C. for 0.5 hours, then heated to reflux at 150° C. for 14 hours to remove water generated from reaction. The solvent was removed via vacuum evaporation and the remaining composition was recrystallized three times by petroleum ether/ethyl acetate to produce the intermediate

DETA-C10-Amine. Yield of the intermediate DETA-C10-Amine was greater than 90.0%.

Synthesis of DETA-C10-SS

An 8.0 wt. % alkali solution was first prepared by dissolving sodium hydroxide (0.5 eq) into H₂O-EtOH (2.5 wt. %:97.5 wt. %). DETA-C10-Amine (1.0 eq), 1,3-propane sultone (0.5 eq) and ethanol were charged into a three-neck round bottle flask and heated at 60° C. for 6 hours. The resulting mixture was cooled down to room temperature, then one-half of the alkali solution was added dropwise into the mixture and stirred for 0.5 hours. An additional 0.25 eq of 1,3-propane sultone was then added at room temperature, and the mixture was heated to 60° C. for another 4 hours. The mixture was then cooled down to room temperature and another 0.25 eq of alkali solution was added. This process was repeated twice by adding 0.125 eq of 1,3-propane sultone and alkali solution separately, and heated at 60° C. until the reaction completed. The mixture was then concentrated and recrystallized 3 times with ethanol/petroleum ether to produce the target product DETA-C10-SS. Yield of DETA-C10-SS was greater than 60.0%.

Synthesis of TETA-C10-Amine Intermediate

TETA (1.0 eq) was dissolved in xylene, then capric acid (2.1 eq) (also dissolved in xylene) was added into the TETA-xylene solution drop by drop at a temperature of 60° C. When completed, orthoboric acid (0.01 eq) was added into the mixture and stirred at a temperature of 100° C. for 0.5 hours, then heated to reflux at 150° C. for 14 hours to remove water generated from reaction. The solvent xylene was removed via vacuum evaporation and the remaining composition was recrystallized 3 times by petroleum ether/ethyl acetate to produce the intermediate TETA-C10-Amine. Yield of the intermediate TETA-C10-Amine was greater than 90.0%.

Synthesis of TETA-C10-SS

An 8.0 wt. % alkali solution was first prepared by dissolving sodium hydroxide (1.0 eq) into H₂O-EtOH (2.5 wt. %:97.5 wt. %). TETA-C10-Amine (1.0 eq), 1,3-propane sultone (1.0 eq) and isopropanol were charged into a three-neck round bottle flask and heated at 60° C. for 6 hours. The resulting mixture was cooled down to room temperature, then one-half of the alkali solution was added dropwise into the mixture and stirred for 0.5 hours. An additional 0.5 eq of 1,3-propane sultone was then added at room temperature, and the mixture was heated to 60° C. for another 4 hours. The mixture was then cooled down to room temperature and another 0.5 eq of alkali solution was added. This process was repeated twice by adding 0.25 eq of 1,3-propane sultone and alkali solution separately, and heated at 60° C. until the reaction completed. The mixture was then concentrated and recrystallized 3 times with isopropanol/petroleum ether to produce the target product TETA-C10-SS. Yield of TETA-C10-SS was greater than 70.0%.

### Thermal Stability

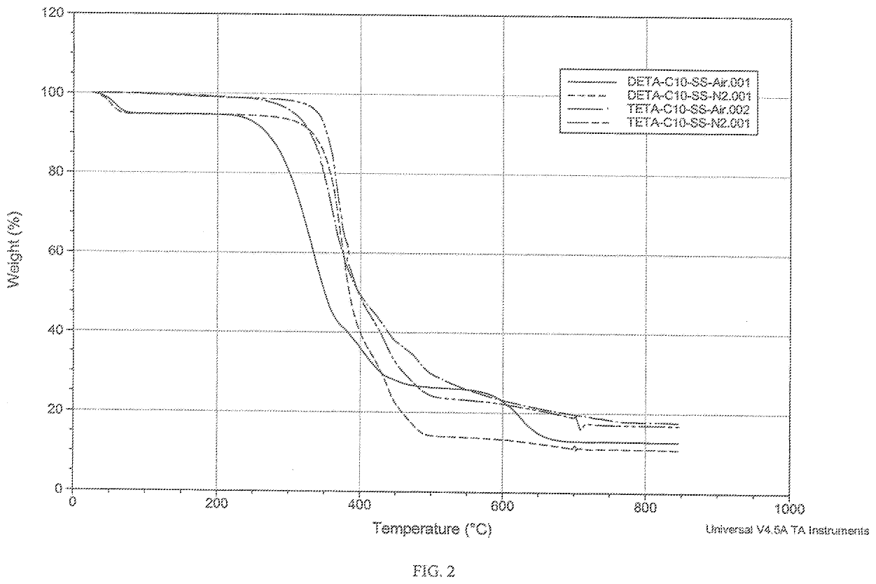

The thermal stability of DETA-C10-SS and TETA-C10-SS were evaluated by thermogravimetric analysis (“TGA”) under air and N₂ atmospheres. As shown in FIG. 2, the surfactants were stable below 200° C., in both air and N₂ atmospheres, and maintained their structural integrity in N₂ atmospheres up to 300° C. These results indicate that the novel surfactants are suitable for high temperature conditions.

### Foaming Performance

Ross-Miles Testing

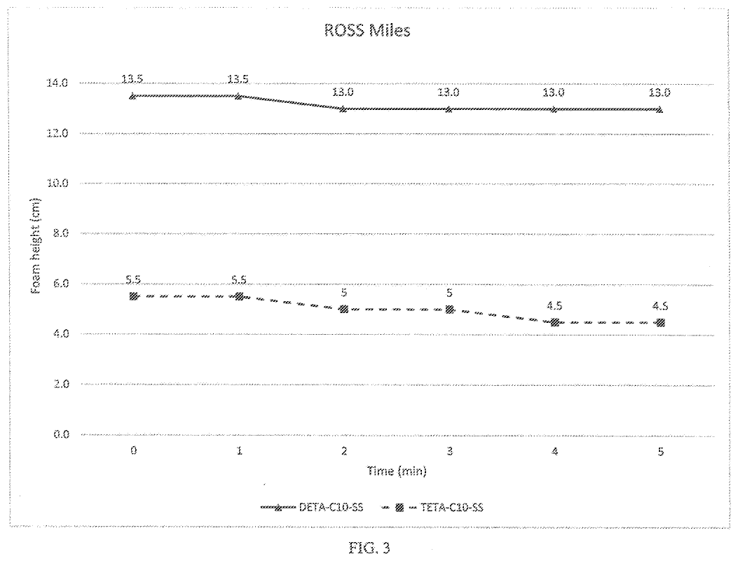

The foamability of the novel surfactants was first measured according to the Ross-Miles method. 200 mL of 0.1 wt. % DETA-C10-SS/DI water solution was poured into a long glass tube, and the foam height was visually checked and recorded every minute. Similarly, 200 mL of 0.1 wt. % TETA-C10-SS/DI water solution was poured into a long glass tube, and the foam height was visually checked and recorded every minute. The results of the testing are depicted in FIG. 3.

As shown in FIG. 3, the initial foam height for DETA-C10-SS was approximately 35 mm, and the foam height dropped to 130 mm after two minutes and maintained its 130 mm height for the next three minutes. Separately, the initial foam height for TETA-C10-SS was 55 mm, and foam height dropped to approximately 45 mm in four minutes.

It is believed that the chemical structure of each of the novel surfactants is responsible for the surfactants' different foamabilities (measured by foam height) and foam stabilities. Specifically, DETA-C10-SS contains two long hydrophobic linear carbon chains, which maximizes foam height. Moreover, DETA-C10-SS contains two amido groups, which can form intermolecular hydrogen bonds to maintain foam stability. In contrast, TETA-C10-SS contains one hydrophilic (CH₂)₃SO₃Na chain, which leads to increased branching and decreases foam stability.

Foam Scanning

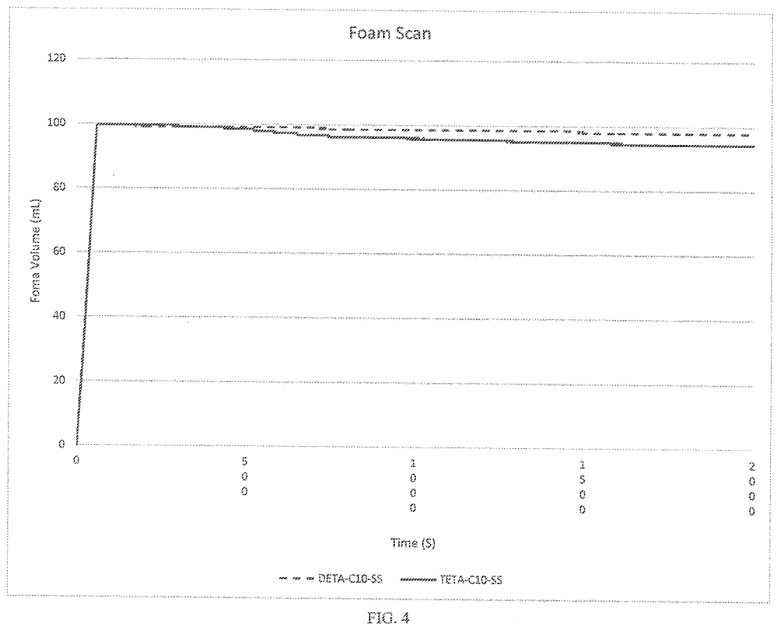

The foaming properties of the novel surfactant solutions were evaluated using a Teclis ITConcept Foamscan device. A 60.0 mL sample of 0.1 wt. % DETA-C10-SS/DI water solution was injected into a reservoir and a constant air flow of 100 mL/min was bubbled into the solution to generate foam. The sample was foamed for 60 seconds after which the foam volume and conductance were measured respectively by the Foamscan program. The procedure was then repeated with a 60 mL sample of 0.1 wt. % TETA-C10-SS/DI water solution. The results of the Foamscan analysis are depicted in FIG. 4.

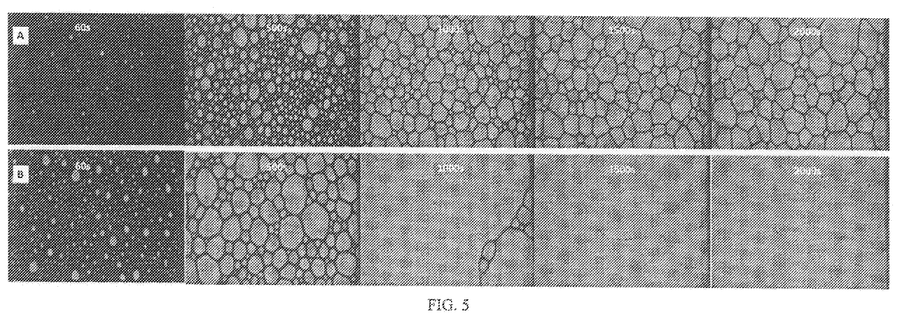

As the data in FIG. 4 shows, the volume of the foam produced by both DETA-C10-SS is approximately 99.7 mL at 60 seconds, and decreases by only 1.8% to 97.9 mL after 2,000 seconds. Similarly, the foam produced by TETA-C10-SS is approximately 99.7 mL at 60 seconds, and decreases by only 5.3% to 94.4 mL after 2,000 seconds. These results confirm that the foams produced by these novel surfactants exhibit superior foam stability to those of surfactant/foams known in the art. The Foamscan device also recorded the morphology of the novel surfactant/foams during testing, which are depicted in FIG. 5 for DETA-C10-SS (Sample A) and TETA-C10-SS (Sample B).

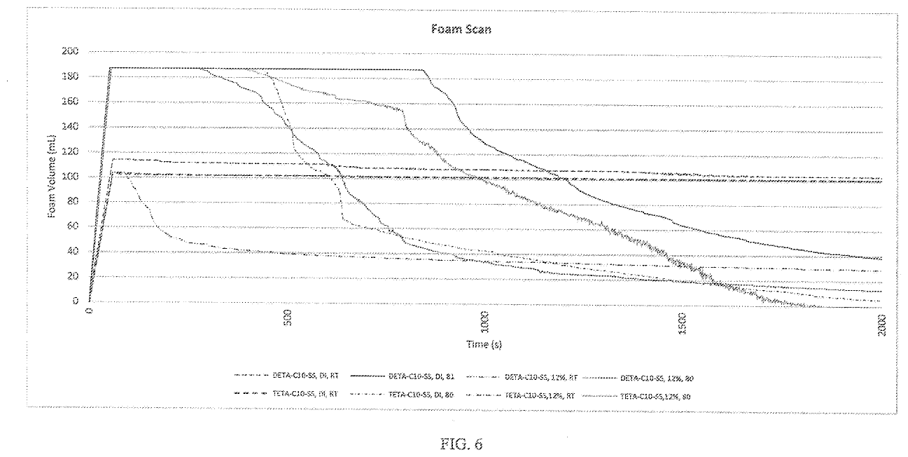

Finally, foam scanning of 0.03 wt. % surfactant solutions of DETA-C10-SS and TETA-C10-SS were evaluated at both an elevated temperature of 80° C., as well as an elevated temperature of 80° C. and a salinity of 12.0 wt. % TDS. Additionally, samples were tested at room temperature, as well as at room temperature and a salinity of 12.0 wt. % TDS. The results, which are depicted in FIG. 6, show that the novel surfactant/foams have superior foam stability at 80° C. compared to foams at room temperature.

Although the invention has been described by reference to its preferred embodiment as is disclosed in the specification and drawings above, many more embodiments of the invention are possible without departing from the invention. Thus, the scope of the invention should be limited only by the appended claims.

1. A method for treating crude oil, said method comprising:
contacting said crude oil with a surfactant in an environment to produce a treated mixture, wherein said surfactant comprises one or more primary components having the chemical formula: 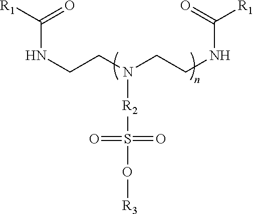
wherein R₁ is a C₅-C₂₁ linear or branched, saturated or unsaturated alkane; R₂ is a C₃-C₁₀ linear or branched, saturated or unsaturated alkane; R₃ is selected from the group consisting of Na, K, Ca, and Mg; and n≥1.

2. The method of claim 1, wherein said surfactant further comprises one or more secondary components having the chemical formula: 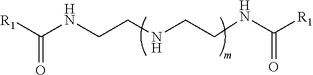
wherein R₁ is a C₅-C₂₁ linear or branched, saturated or unsaturated alkane; and m≥1

3. The method of claim 2, wherein m=n+1

4. A method of synthesizing a surfactant, said method comprising the steps of:
synthesizing a secondary component by reacting an ethyleneamine with a fatty acid, a solvent, and a weak acid.

5. The method of claim 4, further comprising the steps of:
synthesizing a primary component by reacting said secondary component with a sultone compound having a R₂ hydrocarbon chain, an inorganic compound having the formula R₃OH, and an alcohol having the formula R₄OH;
wherein R₂ is a C₃-C₁₀ linear or branched saturated or unsaturated alkane; R₃ is selected from the group consisting of Na, K, Ca, and Mg; R₄ is a C₂-C₃ linear or branched, saturated or unsaturated alkane; and n≥1.

6. The method of claim 5, wherein said ethyleneamine is selected from the group consisting of diethylenetriamine (DETA), triethylene tetramine (TETA), tetraethylene pentamine (TEPA), pentaethylene hexamine (PEHA), hexaethylene heptamine (HEHA) and mixtures thereof.

7. The method of claim 5, wherein said fatty acid is capric acid.

8. The method of claim 5, wherein said solvent is xylene.

9. The method of claim 5, wherein said weak acid is orthoboric acid.

10. The method of claim 5, wherein:
said ethyleneamine has a molecular weight between approximately 100 g/mol. and approximately 325 g/mol.; and
said fatty acid has a molecular weight between approximately 100 g/mol. and approximately 300 g/mol.

11. The method of claim 5, wherein:
said weak acid is between approximately 0.01 wt. % to approximately 2.0 wt. % of the total solution;
said sultone compound having a R2 hydrocarbon chain is between approximately 5.0 wt. % to approximately 35.0 wt. % of the total solution;
said inorganic compound having the formula R₃OH is between approximately 2.0 wt. % to approximately 12.0 wt. % of the total solution; and
said alcohol having the formula R₄OH is between approximately 30.0 wt. % to approximately 70.0 wt. % of the total solution.

12. The method of claim 4, further comprising the steps of:
synthesizing said primary component by reacting said secondary component at 50° C. with C₅H₅N, H₂O and a composition having the chemical formula:

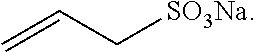

13. The method of claim 4, further comprising the steps of:
synthesizing said primary component by reacting said secondary component with p-Tosyl Chloride, NaOH, Na₂SO₃, NaNO₃, H₂O and a composition having the chemical formula:

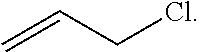

14. The method of claim 4, further comprising the steps of:
synthesizing said primary component by reacting said secondary component with H₂O and a composition having the chemical formula:

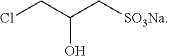

15. The method of claim 4, further comprising the steps of:
synthesizing said primary component by reacting said secondary component at 100° C. with NaOH, H₂O and a composition having the chemical formula:

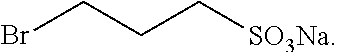

16. A surfactant for treating crude oil, said surfactant comprising one or more first one or more primary components having the chemical formula: 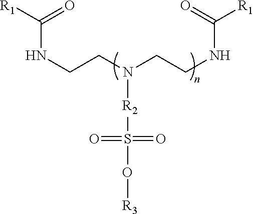
wherein R₁ is a C₅-C₂₁ linear or branched, saturated or unsaturated alkane; R₂ is a C₃-C₁₀ linear or branched saturated or unsaturated alkane; and n≥1.

17. The surfactant of claim 11, wherein said surfactant further comprises one or more secondary components having the chemical formula: 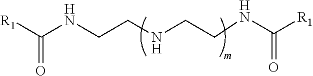
wherein R₁ is a C₅-C₂₁ linear or branched, saturated or unsaturated alkane; and m≥1.

18. The method of claim 1, wherein said primary component has a concentration between approximately 5.0 ppm to 50,000 ppm.

19. The method of claim 2, wherein said secondary component has a concentration between approximately 5.0 ppm to 50,000 ppm.

20. The method of claim 1, wherein the temperature of said environment is between 100° C.-300° C.
